(19) United States
(12) Patent Application Publication
(10) Pub. No.: US 2007/0027549 A1
(43) Pub. Date: Feb. 1, 2007

(54) Gastrointestinal anti-reflux prosthesis apparatus and method

(76) Inventor: Norman Godin, Geneva (CH)

Correspondence Address: COZEN O'CONNOR, P.C., 1900 MARKET STREET, PHILADELPHIA, PA, 19103-3508, US

(21) Appl. No.: 10/570,223

(22) PCT Filed: Sep. 2, 2003
(86) PCT No.: PCT/US03/27414

§ 371 Date: Feb. 28, 2006

(51) Int. Cl. A61F 2/04

(52) U.S. Cl. 623/23.68

(57) ABSTRACT

A prosthesis (13) for implanting in an upper stomach to prevent gastric reflux in an esophagus comprising a tube (17) made of a biocompatible polymer that is resistant to gastric acid, the tube having an upper end and a lower end, a length, and a generally constant diameter along the entire length thereof, the upper end having means (16) for securing to the upper stomach, the lower end having at least one slit (18) to facilitate inversion of the tube during vomiting but to facilitate collapsing of the tube under pressure generated by gastric reflux.

### BACKGROUND OF THE INVENTION

This invention relates to medical devices and their use, and more particularly to implantable prosthesis apparatus which prevent or reduce gastrointestinal reflux.

In my prior U.S. Pat. No. 5,861,036 of Jan. 19, 1999, I disclosed a prosthesis in the form of a flexible tube having a substantially uniform cross section, means for endoscopically placing stitches or clips in a hiatal hernia, and a flexible tubular portion enabling the tube to be squeezed by exerting a pressure on the outer surface thereof in order to prevent reflux of the stomach contents into the esophagus. The prior device was designed so that food could pass freely through the prosthesis in the direction from the esophagus to the stomach. The prosthesis disclosed in my prior patent was made of a biocompatible polymer optionally containing barium sulphate to make it detectable using X-rays.

Although the prior prosthesis worked well in most situations, there were risks that in some cases wherein large chunks of food were swallowed or vomiting occurred, the tube would become dislodged.

In a patent application filed May 20, 2003, PCT/US03/15731, entitled Apparatus And Method For Securing A Device To An Internal Wall Of A Biological Lumen, I disclosed an improved clip design which is intended to prevent the dislodging more effectively than the design disclosed in my aforementioned patent. However, even the improved clip design may not be sufficient in all cases for severe vomiting events, and so further improvements were sought.

### SUMMARY OF THE INVENTION

The present invention comprises, in one aspect, a prosthesis for implanting in an upper stomach to prevent gastric reflux in an esophagus comprising a tube made of a biocompatible polymer that is resistant to gastric acid, the tube having an upper end and a lower end, a length, the upper end having means for securing to the upper opening of the stomach, the lower end having at least one slit to facilitate inversion of the tube during a high pressure vomiting event but to facilitate collapsing of the tube under a level of lateral pressure generated by gastric reflux.

In another aspect, the invention comprises A method of preventing gastric reflux in a patient comprising implanting a prosthesis comprising a tube having an upper end and a lower end, a length, and a generally constant sectional diameter along the entire length thereof by securing the upper end to the upper opening of the patient's upper stomach and allowing the lower end to hang in the upper stomach, the tube having at least one slit at the lower end and adapted to collapse under a level of lateral pressure generated by gastric reflux from the stomach, invert during a high level of lateral pressure generated by vomiting so as to permit vomit to exit, and upon reduction of pressure at the completion of the vomiting, the tube to return to the pre-vomiting position.

It is preferable that the prosthesis have one to eight slits, and most preferable that it have two slits.

The tube in some embodiments may have a constant sectional diameter along the entire length thereof from the upper end to the lower end, with the slits having a length of at least about one third of the length of the tube from the lower end toward the upper end. The slits in other embodiments may have a length of between 66% and 95% of the length of the tube.

The tube should be made of the biocompatible polymer, for example medical grade polyurethane, silicone, or polystyrene-ethylene (PSE).

The length of the tube, from upper end to lower end, is preferably about 2 to 10 cm.

As disclosed in my aforementioned PCT application, the prosthesis may be secured to the upper opening of the stomach with clips, preferably at least three clips, which are opened and closed with an endoscopic device.

The advantages of the slit valve are that larger pieces of food can be swallowed by the patient without having the food getting caught in the tubular valve. The valve allows vomiting at lower pressures, such as about 50 mm of Hg of back pressure and once the valve is reverted, it is easier for the valve to resume its original position once the patients drinks fluids or eats something, therefore eliminating the risk of food being blocked by the valve after vomiting.

As is the case with the prosthesis of my prior inventions, the present prosthesis is a soft tubular element of a fairly constant section which win prolong the esophagus into the stomach. As the stomach has an asymmetrical shape in relationship to the axis of the esophagus, in case of gastric reflux, the exerted pressure has a oblique direction in relationship to the axis of the esophagus. Therefore, if one prolongs the esophagus with a soft tube extending for a certain length into the stomach, in case of gastric reflux the soft tube collapses under the oblique pressure and stops the exit of gastric acid into the esophagus. The tube polymer must be resistant to gastric acids. The dimensions of the tube may vary, but preferably the tube has a section diameter between 25 and 30 millimeters from one end to the other and a length of between 5 and 10 centimeters, with a thickness of the wall chosen to allow it to collapse under lateral pressure applied to its external wall, when the level of pressure generated by gastric reflux is reached, generally between approximately 0.2 and 0.6 mm.

### BRIEF DESCRIPTION OF THE DRAWINGS

FIG. 1 is a sectional view of a patient's stomach and esophagus with a balloon catheter.

FIG. 2 is a sectional view of a patient's stomach and esophagus with the prosthesis according to the invention being inserted.

FIG. 3 is a sectional view a patient's stomach and esophagus with the prosthesis according to the invention in place and about to be clipped to the esophagus wall.

FIG. 4 is a sectional view a patient's stomach and esophagus with the prosthesis according to the invention clipped to the esophagus wall.

FIG. 5 is sectional view a patient's stomach and esophagus with the prosthesis according to the invention clipped to the esophagus wall and resisting normal gastric pressures shown by arrow 20.

FIG. 6 sectional view a patient's stomach and esophagus with the prosthesis according to the invention clipped to the esophagus wall with the tube reversed inside out due to large gastric vomiting pressure indicated by arrow 24.

FIG. 7 is a sectional view a patient's stomach and esophagus with the prosthesis according to the invention clipped to the esophagus wall with the tube in its normal position, with large chunks of undigested food 21 passing through.

FIG. 8 is a sectional view of a patient's stomach and esophagus with the prosthesis of the present invention unclipped and being removed by a hook on a removal device 11 on a basket type handle 22 on the top of the prosthesis.

### DESCRIPTION OF THE PREFERRED EMBODIMENTS

While many different embodiments of the invention are contemplated and are possible, one embodiment is illustrated herein and in the drawings.

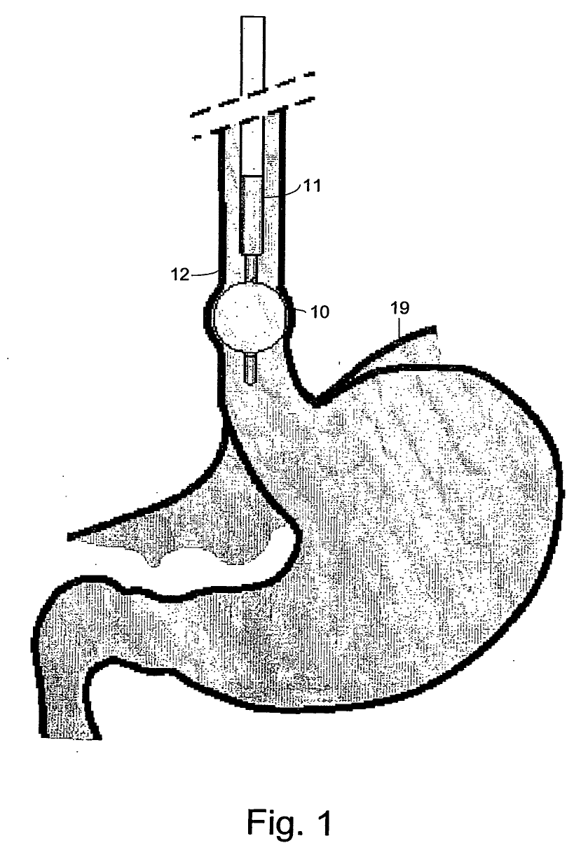

Referring first to FIG. 1, a stomach 19 and esophagus 12 are shown with insertion device 11 carrying a balloon catheter 10 to widen the esophagus prior to insertion of the prosthesis of the invention.

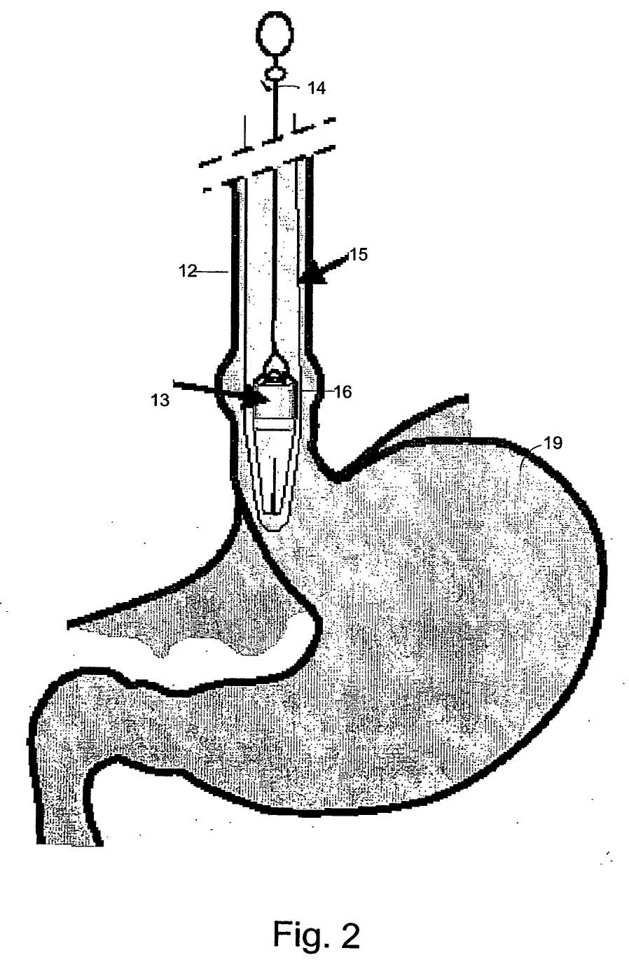

FIG. 2 shows forceps 14 operating delivery-retrieval wire 15 installing the prosthesis 13, with the clips 16 shown closed.

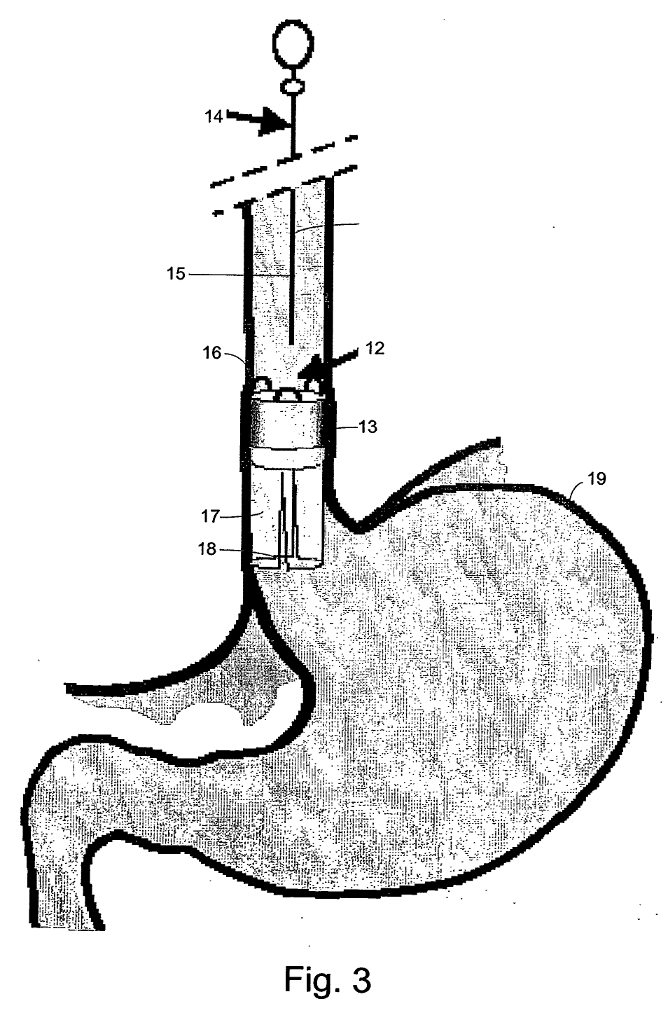

FIG. 3 shows forceps 14, wire 15, clips 16, with the prosthesis 13 about to be installed at the top of the upper stomach 19 by means of clips 16. The slits 18 in tube 17 are illustrated in this view.

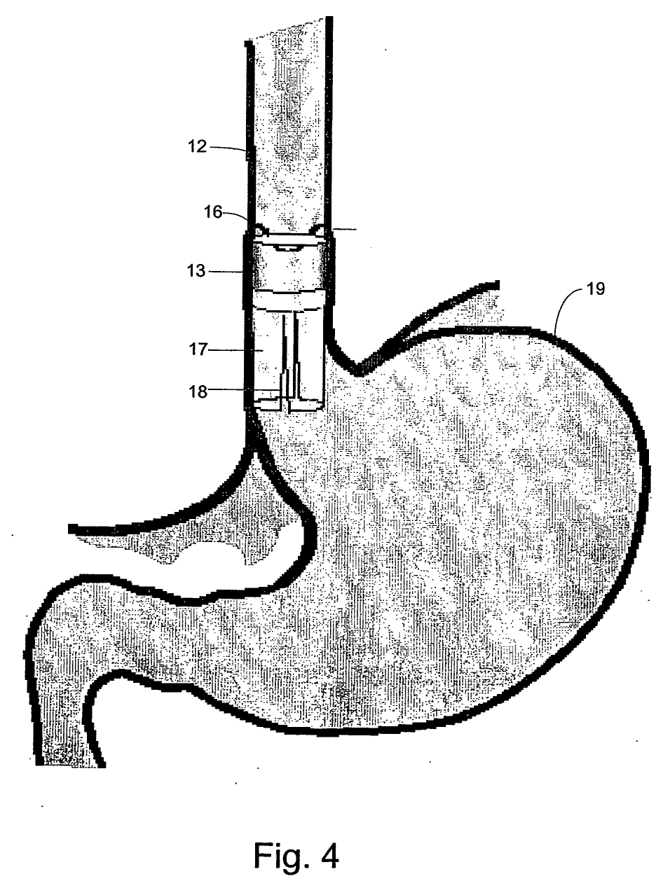

FIG. 4 shows the prosthesis 13 with clips 16 installed in the wall of the esophagus at the top of upper stomach 19, with tube 17 hanging into upper stomach 19, with slits 18 shown.

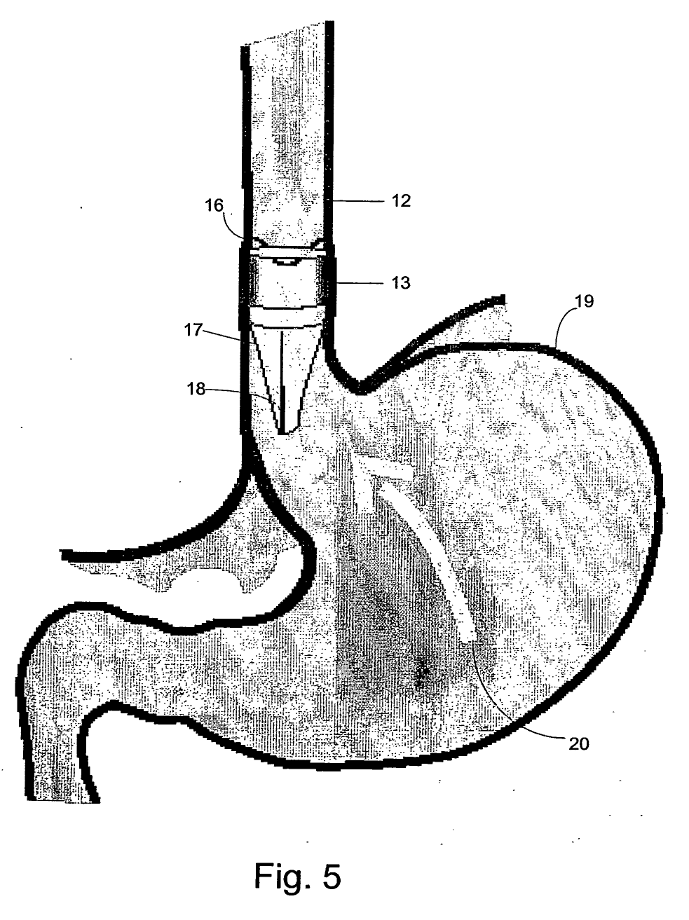

FIG. 5 shows the tube 17 being closed when gastric reflux pressure 20 occurs. The force has an oblique direction in relationship to the long axis of the tube so that with a flexible wall, the tubular prosthesis will collapse against the stomach wall and will stop the gastric acid from leaving the stomach and reaching the mucosa of the esophagus.

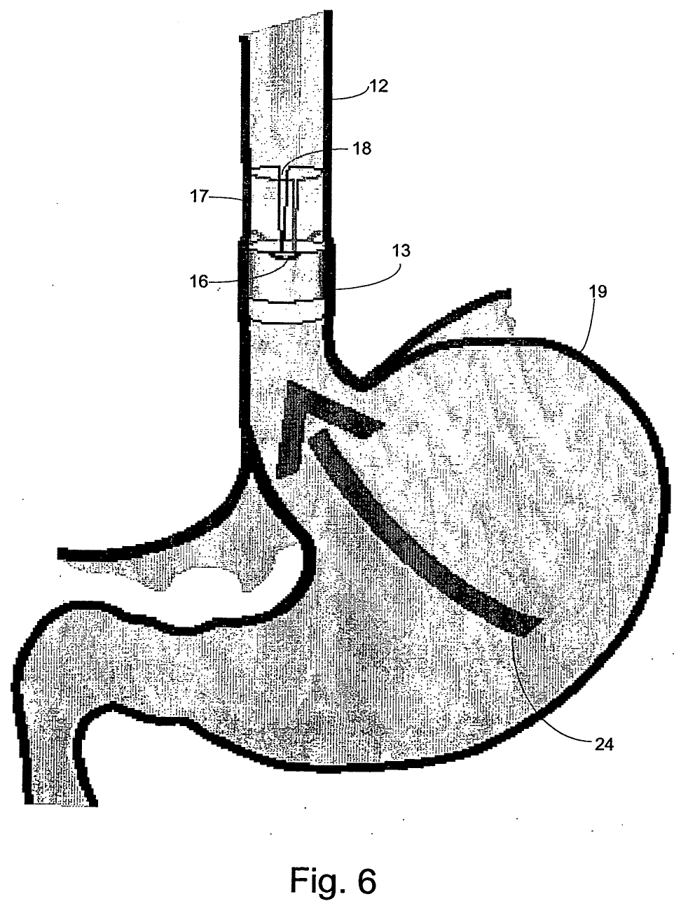

FIG. 6 shows the tube 17 reversing inside out into the esophagus when intense vomiting pressure, shown by large direction arrow 24, occurs.

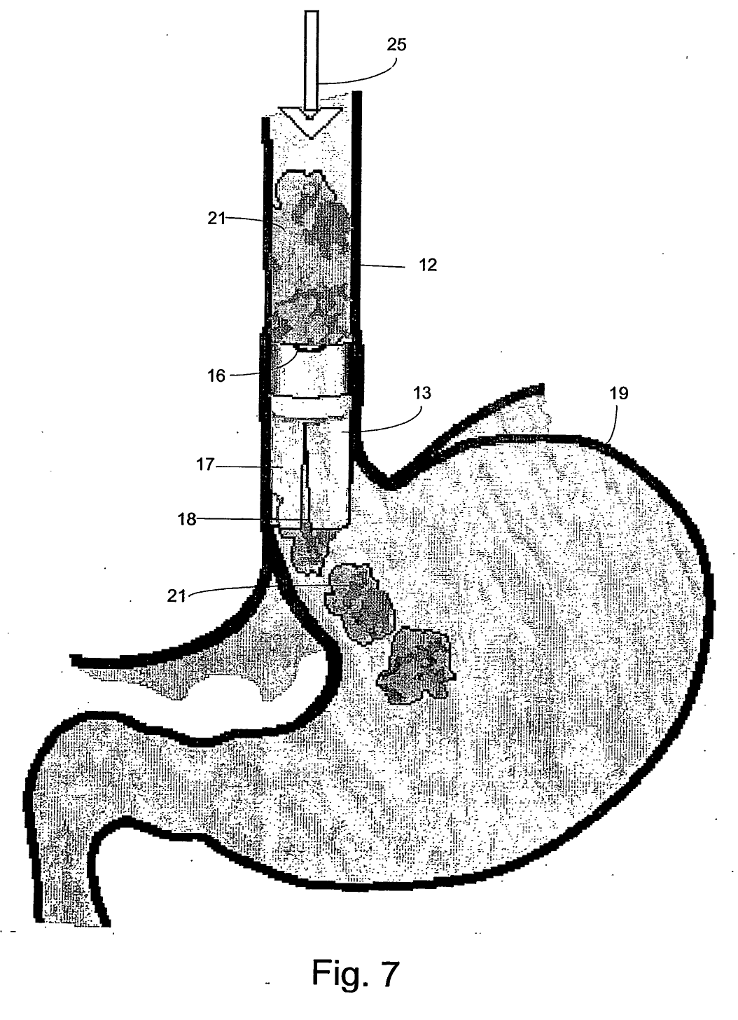

FIG. 7 shows the tube 17 in its normal position in the upper stomach when large chunks of food 21 are being swallowed in direction 25 and going through the prosthesis tube 17, which is made easier and more efficient due to the slits 18.

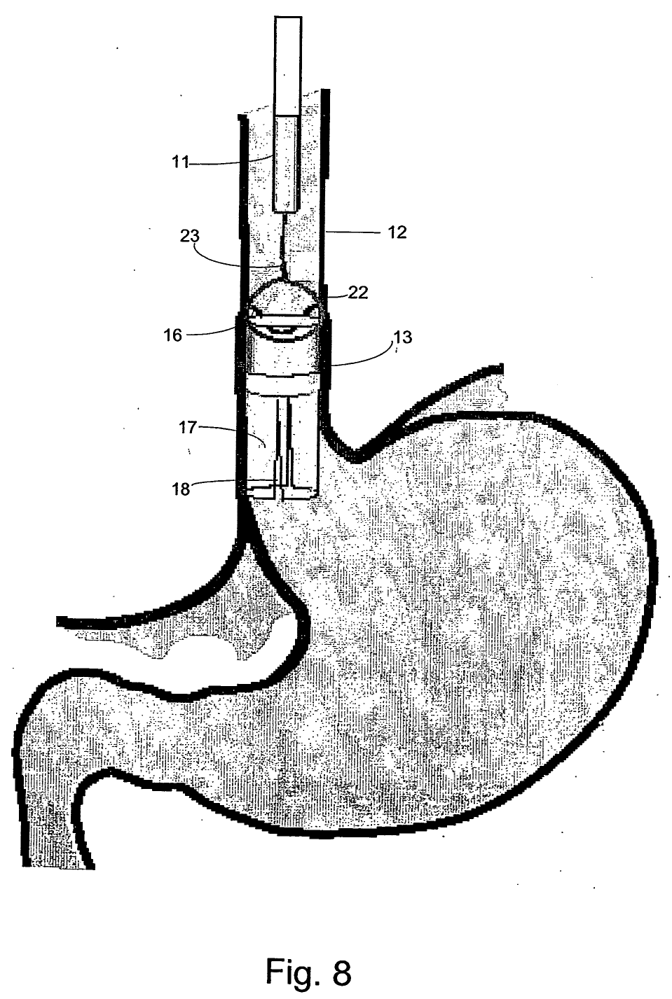

FIG. 8 shows the prosthesis 13 being unclipped and removed by means of removal device 11, using its hook 23 on prosthesis handle 22, after unclipping clips 16 from the esophagus wall 12.

The tubular prosthesis 17 can be manufactured using several different techniques depending on the biomaterial used, particularly depending on the viscosity of the biomaterial and the thickness of the wall of the prosthesis. The prosthesis can be manufactured by injection molding, extrusion molding or by solvent casting, which is a method of dipping the prosthesis in a solution of the biomaterial until the desired thickness of the wall is obtained. The slits are manufactured after the tube is formed.

While the invention has been described and illustrated in detail, various modifications, alternative embodiments, and improvements should become readily apparent to those skilled in this art without departing from the spirit and scope of the invention.

1. A prosthesis for implanting in an upper stomach to prevent gastric reflux in an esophagus comprising a tube made of a biocompatible polymer that is resistant to gastric acid, the tube having an upper end and a lower end, a length, and a generally constant sectional diameter along the entire length thereof from the upper end to the lower end, the upper end having means for securing to the upper opening of the stomach, the lower end having at least one slit to facilitate inversion of the tube during a high pressure vomiting event but to facilitate collapsing of the tube under a level of lateral pressure generated by gastric reflux.

2. The prosthesis of claim 1 having one to eight slits.

3. The prosthesis of claim 1 having two slits.

4. The prosthesis of claim 1 wherein the tube has a generally constant sectional diameter along the entire length thereof from the upper end to the lower end.

5. The prosthesis of claim 1 wherein the one or more slits have a length of at least about one third of the length of the tube from the lower end toward the upper end.

6. The prosthesis of claim 1 wherein the one or more slits have a length of between 66% and 95% of the length of the tube.

7. The prosthesis of claim 1 wherein the biocompatible polymer is medical grade polyurethane, silicone, or polystyrene-ethylene (PSE).

8. The prosthesis of claim 1 having a length from upper end to lower end of about 2 to 10 cm.

9. The prosthesis of claim 1 wherein the means for securing to the upper opening of the stomach is a set of at least three clips.

10. A method of preventing gastric reflux in a patient comprising implanting a prosthesis comprising a tube having an upper end and a lower end, a length, and a generally constant sectional diameter along the entire length thereof by securing the upper end to the upper opening of the patient's upper stomach and allowing the lower end to hang in the upper stomach, the tube having at least one slit at the lower end and adapted to collapse under a level of lateral pressure generated by gastric reflux from the stomach, invert during a high level of lateral pressure generated by vomiting so as to permit vomit to exit, and upon reduction of pressure at the completion of the vomiting, the tube to return to the pre-vomiting position.

11. The method of claim 10 comprising providing the tube with between 1 and 8 slits.

12. The method of claim 10 comprising providing the tube with 2 slits.

13. The method of claim 10 wherein the one or more slits have a length of about one third the length of the tube.
